SEQUENCE LISTING (13 sequences)

SEQ ID NO: 1 - DNA, 25 bp, Saccharomyces sp.
ctgaatatgcattgaaataagatcc

SEQ ID NO: 2 - DNA, 25 bp, Saccharomyces sp.
atatgcattgaaataagatccaaac

SEQ ID NO: 3 - DNA, 25 bp, Saccharomyces sp.
gcattgaaataagatccaaacagct

SEQ ID NO: 4 - DNA, 25 bp, Saccharomyces sp.
tgaaataagatccaaacagctaaga

SEQ ID NO: 5 - DNA, 25 bp, Saccharomyces sp.
ataagatccaaacagctaagaacag

SEQ ID NO: 6 - DNA, 25 bp, Saccharomyces sp.
gatccaaacagctaagaacaggaaa

SEQ ID NO: 7 - DNA, 45 bp, Saccharomyces sp.
gacttatacgtaactttattctatgtttgtcgattcttgtccttt

SEQ ID NO: 8 - DNA, 16 bp, Saccharomyces sp.
ttcaaatgggtgaaca

SEQ ID NO: 9 - DNA, 16 bp, Saccharomyces sp.
ttccaatgggtgaaca

SEQ ID NO: 10 - DNA, 16 bp, Saccharomyces sp.
ttacaatgggtgtgca

SEQ ID NO: 11 - DNA, 16 bp, Saccharomyces sp.
ttccaatgggtgaaca

SEQ ID NO: 12 - DNA, 16 bp, Saccharomyces sp.
ttcaaatgggtgaaca

SEQ ID NO: 13 - DNA, 16 bp, Saccharomyces sp.
ttccaatgggtgaaca

(19) United States
(12) Patent Application Publication
(10) Pub. No.: US 2009/0264307 A1
(43) Pub. Date: Oct. 22, 2009

(54) ARRAY-BASED POLYMORPHISM MAPPING AT SINGLE NUCLEOTIDE RESOLUTION

(75) Inventors: David Gresham, Princeton, NJ (US); David Botstein, Princeton, NJ (US); Leonid Kruglyak, Princeton, NJ (US); Douglas Ruderfer, Cambridge, MA (US); Stephen Pratt, Phoenix, AZ (US)

Correspondence Address: DUANE MORRIS LLP - Philadelphia;IP DEPARTMENT, 30 SOUTH 17TH STREET, PHILADELPHIA, PA, 19103-4196, US

(73) Assignee: The Trustees of Princeton University, Princeton, NJ (US)

(21) Appl. No.: 12/160,266

(22) PCT Filed: Jan. 12, 2007
(86) PCT No.: PCT/US07/00987

§ 371 Date: Nov. 19, 2008

Related U.S. Application Data
(60) Provisional application No. 60/759,099, filed on Jan. 13, 2006.
(60) Provisional application No. 60/779,613, filed on Mar. 6, 2006.

(51) Int. Cl. C40B 30/04; C12Q 1/68; C40B 40/06

(52) U.S. Cl. 506/9; 435/6; 506/16

(57) ABSTRACT

Disclosed herein is a system useful for detecting sequence differences (e.g., single-nucleotide polymorphisms) between genomes using data from a single hybridization with a genomic DNA microarray, such as a whole-genome array. The methods described herein can be used to detect, simply and inexpensively, differences in sequence among the genomes of individual members of a species, for example. In examples described herein, the system and methods were used to detect a variety of spontaneous single base-pair substitutions, insertions and deletions, and most (>90%) of the approximately 30,000 known single-nucleotide polymorphisms between two Saccharomyces cerevisiae strains. The system and methods were also used to elucidate the genetic basis of phenotypic variants and identify the small number of single base-pair changes accumulated during experimental evolution of yeast.

### BACKGROUND OF THE DISCLOSURE

The disclosure relates generally to the field of nucleotide sequencing and comparison of nucleotide sequences.

Despite the ongoing development of DNA sequencing technology, it remains technically and financially infeasible for individual laboratories to sequence whole genomes (Margulies et al., 2005, Nature 437:376; Shendure et al., 2005, Science 309:1728). Moreover, determining the entire genomic sequence is unnecessary when making global comparisons of genomes within a species, because one expects a relatively small number of sequence differences throughout the genome. In such cases, it is sufficient to assess the extent and location of sequence variation, for example in a manner analogous to comparative genomic hybridization (CGH), which compares copy number changes between two related genomes at genic resolution (Pollack et al., 2002, Proc. Natl. Acad. Sci. USA 99:12963).

DNA microarrays of short oligonucleotides designed to interrogate each base individually (i.e., resequencing arrays) have been applied to the analysis of individual human genes and small genomes such as the human mitochondrial genome and the SARS coronavirus genome (Hacia et al., 1996, Nat. Genet. 14:441; Maitra et al., 2004, Genome Res. 14:812; Wong et al., 2004, Genome Res. 14:398). However, extension of this approach to whole genomes is currently impractical for most organisms, owing to the large number of probes that would be required for complete coverage.

An alternative approach uses microarrays that detect mismatches, exploiting the fact that hybridization to a short oligonucleotide is quantitatively very sensitive to the number and position of mismatches (Maskos et al., 1992, Nucl. Acids Res. 20:1675). Sequence-level differences are detected, without allele-specific probes, by comparing hybridization intensities of individual features on the microarray (referred to as single feature polymorphisms {SFPs}; Winzeler et al., 1998, Science 281:1194). This method has been successfully applied to studies of genetic diversity and gene mapping, for example (Turner et al., 2005, PLoS Biol. 3:e285; Volkman et al., 2002, Science 298:216; Winzeler et al., 2003, Genetics 163:79; Borevitz et al., 2003, Genome Res. 13:513; Brem et al., 2002, Science 296:752; Deutschbauer et al., 2005, Nat. Genet. 37:1333; Ronald et al., 2005, PLoS Genet. 1:e25; Steinmetz et al., 2002, Nature 416:326; Yvert et al., 2003, Nat. Genet, 35:57).

Until recently, comprehensive detection of single base-pair differences has been limited by probe density across an organism's genome, which is typically a few oligonucleotides per gene. Even complete single-copy coverage is unlikely to be sufficient for finding all mutations, since statistically detectable decreases in hybridization intensity usually require that a variant nucleotide fall within the central 15 bases of a 25-basepair (bp) probe (Ronald et al., 2005, Genome Res. 15:28).

The technology disclosed herein overcomes these difficulties and shortcomings and provides methods of assessing polymorphisms in whole genomes of individual organisms.

### BRIEF SUMMARY OF THE DISCLOSURE

The disclosure relates to a method of assessing the likelihood that a polymorphism occurs at a nucleotide position in a first genome, relative to the same nucleotide position in a corresponding reference genome. The method comprises contacting, under hybridizing conditions, fragments of the first genome with a plurality of polynucleotide probes. The probes are completely complementary to different, but overlapping, portions of the reference genome that include the nucleotide position. One then assesses the hybridization intensity for each of the probes with the fragments. For each probe, the assessed hybridization intensity is compared with the predicted hybridization intensity. The predicted hybridization intensity for each probe is calculated based on hybridization intensity of fragments of the reference genome with the probes. The comparisons for each probe are compared to yield an estimate the polymorphism occurs at the nucleotide position in the first genome.

In one embodiment of this method, the predicted hybridization intensity for each probe is further calculated based on assumed occurrence of a non-complementary nucleotide residue, at the residue corresponding to the nucleotide position within the probe.

The likelihood that a polymorphism occurs can be assessed at each of a plurality of closely-spaced nucleotide positions in the first genome, and a polymorphism is predicted to occur at the position having the highest estimate that the polymorphism occurs at or near that position. In another embodiment, the likelihood that a polymorphism occurs is assessed at each of a plurality of (e.g., 10, 50, or 1000) consecutive nucleotide positions in the first genome, and a polymorphism is predicted to occur at the position having the highest estimate that the polymorphism occurs at that position.

The fragments of the first genome can be prepared by enzymatic digestion, such as by digestion with DNaseI. Alternatively, the fragments can be prepared by amplification of portions of the first genome. Preferably, substantially all of the fragments of the first genome have a length in the range from 15 to 50 nucleotide residues.

Each of the probes can be bound to a support at a discrete location in the methods described herein. For example, each of the probes can be bound to a discrete support. Alternatively, the fragments can be bound to a support at discrete locations.

In the methods described herein, hybridization intensity can be assessed, for example, by assessing fluorescence of a dye associated with the probes. By way of example, the probes can be biotinylated and the dye can be bound (i.e., linked to) with an avidin.

Predicted hybridization intensity for each probe can be calculated based both on the occurrence of a non-complementary nucleotide residue at the residue corresponding to the nucleotide position within the probe and on the occurrence of a complementary nucleotide residue at the residue corresponding to the nucleotide position within the probe. The predicted hybridization intensity for each probe can corrected for the hybridization intensity variance observed for a plurality of hybridizations performed using fragments of the reference genome. Similarly, the predicted hybridization intensity for each probe can take into account the G-C content of the probe, the cross-hybridization potential of the probe, and the tendency of the probe to form secondary structure that impedes hybridization.

In a convenient way of performing the methods described herein, the comparisons of assessed and predicted hybridization intensities are made by calculating a prediction signal (Lₖ) from the values obtained for each of i probes. For example, such Lₖ values can calculated using the following formula.

$${L}_{k} = ( {log}_{10} e ) {\sum }_{i} \frac{{( {x}_{i} - {μ}_{ni} )}^{2} - {( {x}_{i} - {μ}_{pi} )}^{2}}{2 {σ}_{i}^{2}}$$

In this formula, xᵢ is the assessed hybridization intensity for an individual probe, μₙᵢ is the predicted hybridization intensity for the individual probe when no polymorphism occurs at the nucleotide position, μₚᵢ is the predicted hybridization intensity for the individual probe when a polymorphism occurs at the nucleotide position, and σᵢ is the intensity variance for the probe. A positive value of Lₖ is indicative that a polymorphism occurs at the nucleotide position and a negative value of Lₖ is indicative that a polymorphism does not occur at the nucleotide position. In this method, μₙᵢ can be assessed based on hybridization intensity values observed when fragments of the reference genome are hybridized with the probe. The value of σᵢ can be assessed based on the variance in hybridization intensity values observed when fragments of the reference genome are hybridized with the probe. Furthermore, μₚᵢ can be calculated for each of the i probes using the formula μₚᵢ=μₙᵢ−Dᵢ. In that formula, Dᵢ can be calculated using this second formula.

Dᵢ=α+β(GC)+γ(PMᵢ−MMᵢ)+δPMᵢ

In this second formula, Dᵢ is the signal difference for a completely complementary probe overlapping a polymorphism, GCᵢ is the G-C content of the probe, PMᵢ is the hybridization intensity of the probe when hybridized with a completely complementary polynucleotide, MMᵢ is the hybridization intensity of the probe when hybridized with a complementary polynucleotide having a single mismatched base at its center, and each of α, β, γ, and δ is a constant derived from statistical fitting of data obtained from hybridization of the probe with fragments of the reference genome.

The methods described can be used with a variety of genomic sequence. For example, the sequence of the reference genome can be one that is substantially known. The first and reference genomes can be genomes of a normally haploid organism, genomes of a homozygous diploid organism, homozygous portions of the genome of a diploid organism, or haploid portions of the genome of a diploid organism, for example. In one embodiment, the first and reference genomes are either the Y chromosome or the X chromosome of a human. In another embodiment, the first and reference genomes are the genomes of a subcellular organelle (e.g., a mitochondrion or a chloroplast) of a eukaryote. In embodiments described in the examples herein, the first and reference genomes are the genomes of a yeast. The first and reference genomes can be the genomes of one of a bacterium and an archaebacterium, or genomes obtained from different strains of the same species.

Preferably, the probes used in the methods described herein have lengths in the range from 10 to 60 (preferably from 25 to 45 or from 25 to 35) nucleotide residues. The assessed and predicted hybridization intensities are preferably not compared for probes for which the probe residue corresponding to the nucleotide residue occurs within 5 residues from an end of the probe.

The methods described herein can be used to assess the location of one or more polymorphisms in a first genome, relative to a corresponding reference genome. This end can be achieved by assessing the likelihood that a polymorphism occurs at a nucleotide position in the first genome, relative to the same nucleotide position in the reference genome for each of a plurality of genomic locations which are overlapped by multiple probes. The estimates for the plurality of locations are compared to identify where, within the overlapping portions, a polymorphism occurs. The polymorphism is predicted to occur at the nucleotide residue for which the estimate is highest.

The disclosure also relates to a kit for assessing occurrence of one or more polymorphisms in a first genome, relative to a corresponding reference genome. The kit includes at least two elements. First, the kit includes an array that comprises a plurality of polynucleotide probes attached at discrete known locations on a support. The probes are completely complementary to different, but overlapping, portions of the reference genome. Second, the kit includes a digital storage medium that includes an algorithm for comparing assessed hybridization intensity values for the probes with predicted hybridization intensity values. Occurrence of one or more polymorphisms in the first genome can be assessed using this kit.

### BRIEF SUMMARY OF THE SEVERAL VIEWS OF THE DRAWINGS

FIG. 1 is a diagram that illustrates the decrease in intensity observed at each probe as function of SNP position. The greatest decrease in intensity was observed when the polymorphism fell within the central ten bases of the probe. The end of each box marks the upper and lower quartiles, and the filled circle within each box indicates the median value. The brackets extend to the furthest data point no more than 1.5 times the interquartile range from the box. Empty circles show outlier values.

FIG. 2 is a diagram showing that the difference in intensity between a probe and its corresponding mismatch probe (the horizontal axis represents Perfect match (PM)−mismatch (MM) intensity) is linearly related to the decrease observed at a PM probe when an experimental DNA sample containing a SNP is hybridized to the yeast tiling microarray YTM (vertical axis). The end of each box marks the upper and lower quartiles, and the filled circle within each box indicates the median value. The brackets extend to the furthest data point no more than 1.5 times the interquartile range from the box. Empty circles show outlier values.

FIG. 3 is a graph which illustrates that by increasing the threshold prediction signal score (indicated on the graph) at which a SNP is called, the number of false positives is minimized while retaining a high true positive rate in a test set of 981 RM-11-1a SNPs (dashed line).

FIG. 4 is a graph that depicts the proportion of SNPs correctly predicted and the number of false positive predictions obtained using the SNPscanner algorithm at various prediction signal threshold values. The SNPscanner algorithm was used to generate a nucleotide-level comparison of a genome divergent from the sequenced reference genome. The proportion of the 30,303 known SNPs in the yeast strain YJM789 that were detected using the algorithm is shown on the vertical axis. Numbers on the graph indicate prediction signal thresholds. Using data from a single hybridization experiment, we were able to correctly identify as many as 28,737 SNPs (at a prediction signal threshold of 1). At prediction signal threshold value of 5, the number of false positive predictions was reduced to 8 in a test of the reference genome and 86.9% of true positives were still predicted.

FIG. 5 consists of FIGS. 5A, 5B, 5C, and 5D. The data in this figure demonstrate that the SNPscanner algorithm accurately predicts SNPs in CAN1 for independent yeast CAN^{R} mutants. FIG. 5A is an alignment of multiple overlapping probes (SEQ ID NOs: 1-6) that are complementary to a site (SEQ ID NO: 7) in the CAN1 gene. A mutation at the site indicated in bold, underlined text perturbs hybridization of the sample to each of the probes depicted. The decrease in observed hybridization (relative to hybridization of a perfectly-matched probe) is used to estimate the likelihood of occurrence of a polymorphism at the site. FIG. 5B is a graph that illustrates the prediction signal generated from hybridization of genomic DNA of a single yeast CAN^{R} mutant having a G-->T base substitution predicted at chromosomal coordinate 31843. FIG. 5C is a graph of the prediction signal generated using the SNPscanner algorithm to detect single base-pair substitutions and a 1 bp deletion in four independent spontaneous CAN^{R} yeast mutants isolated in a reference genome background. FIG. 5D is a graph of the prediction signal generated using the SNPscanner algorithm to predict mutations and SNPs in a non-reference genome. The signals for nine independent CAN^{R} mutants in the CEN.PK strain background are shown for the entire CAN1 gene. We confirmed unique nucleotide substitutions for seven of the mutants and a single base insertion in one mutant and a single base deletion in another. At common polymorphisms, the SNPscanner signal is highly reproducible across multiple samples allowing intra-strain comparisons of non-reference genomes.

FIG. 6 is a graph of the prediction signal at various chromosomal coordinates. A single mutation was predicted in the 1809 bp GAP1 gene corresponding to a C→G mutation at nucleotide 514,919 on chromosome XI (peak indicated). The confirmed mutation falls within GAP1, which is shown in its entirety.

FIG. 7 is a graph of prediction signal at various chromosomal coordinates. A single mutation was predicted in the 477 bp FCY1 gene corresponding to a C→T mutation at nucleotide 677,256 on chromosome XVI (peak indicated). The confirmed mutation is within FCY1, which is shown in its entirety.

FIG. 8 is a graph showing the physical locations of hybridized digested genomic DNA associated with the Affymetrix YTM microarray. Visual inspection of the physical location of probes associated with predictions of polymorphisms revealed likely manufacturing artifacts on some microarrays. To reduce false positives due to such defects on the YTMs, probes that showed an obvious geographical clustering, such as that indicated by the arrow, were excluded.

FIG. 9 is a graph of prediction signal at various chromosomal coordinates across the entire region of chromosome 1 of the laboratory strain, CEN.PK. CEN.PK is a mosaic of regions completely isogenic to S288C, as indicated by large regions of prediction signal below zero, regions of high nucleotide divergence, and large deleted sections detectable by continuous regions of positive prediction signal.

FIG. 10 is a graph of the prediction signal generated using the SNPscanner algorithm for genome-wide mutation detection. Whole genome analysis of a strain in which AMN1 was deleted but failed to demonstrate the expected non-clumpy phenotype predicted the presence of a 1,562 bp deletion in ACE2 (shown in its entirety; chrXI:404,511-406,823). Sequence analysis confirmed the deletion, which spans 1558 bp and is flanked by the nucleotide sequence CTG, and mapped the breakpoints to nucleotides 404,621-406,179.

### DETAILED DESCRIPTION

The disclosure relates to methods of assessing occurrence of sequence polymorphism in genomic DNA. The methods can be used to identify the location of such polymorphisms without a priori knowledge of their sequence or location and do not require sequencing of the genomic DNA.

There are many known applications for technologies such as those described herein. By way of example, these methods can be used for targeted “resequencing” of known genomic sequence without the need for base-by-base sequencing methods. Instead, the methods described herein can be used to detect changes throughout the genome relative to a known reference sequence in a single hybridization procedure. Detection of such changes can be of critical importance in applications such as identification of mutations (e.g., mutations conferring drug resistance to a viral or microbial pathogen) and characterization of isolated organisms (e.g., identification of the species and/or strain of a pathogenic agent for the purpose of selecting a therapeutic agent known to be efficacious therefor).

### DEFINITIONS

As used herein, each of the following terms has the meaning associated with it in this section.

The term “hybridization intensity” refers to any quantitative measure of nucleic acid hybridization. The term “hybridization” refers to the well-known process by which two single-stranded polynucleotides non-covalently associate under appropriate conditions to form a stable double-stranded helical structure. This quantity is commonly (and in the examples disclosed herein) assessed by labeling at least one of the nucleic acids being hybridized with a detectable label and, after subjecting the nucleic acids to hybridization conditions, assessing the quantity of the label that is present in hybridized nucleic acids (e.g., fluorescence intensity of fluorescently-labeled nucleic acids that are hybridized with nucleic acids bound to a support). The phrase includes any measure of nucleic acid hybridization, regardless of the detection or labeling method.

Methods for conducting polynucleotide hybridization assays have been well developed in the art. The term “hybridizing conditions” refers to any set of chemical and physical parameters that facilitate hybridization between complementary polynucleotides that are permitted to contact one another. Hybridization assay procedures and conditions will vary depending on the application and are selected in accordance with the general binding methods known including those referred to in, for example, Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young et al., P.N.A.S, 80: 1194 (1983). Methods and apparatus for carrying out repeated and controlled hybridization reactions have been described, for example, in U.S. Pat. Nos. 5,871,928; 5,874,219; 6,045,996; 6,386,749; and 6,391,623. Hybridizing conditions will commonly include salt concentrations of less than about 1M. Other factors (e.g., base composition, concentration, and length of the complementary strands, presence of organic solvents, hybridization temperature, degree of agitation, and extent of base mismatching) can affect hybridization. As a result, the combination of parameters is more important than the absolute measure of any one alone. Selection of appropriate hybridizing conditions is within the skill of an ordinary artisan in this field. The conditions disclosed herein for hybridization of nucleic acids can be used as a guide if desired, but are merely examples of suitable conditions.

With regard to a sample genomic DNA being analyzed according to the methods described herein, a “corresponding reference genome” refers to a genome (or a portion thereof) for which at least some sequence homology with the sample genome is known, expected, or suspected. By way of example, if genomic DNA of a particular strain of Saccharomyces cerevisiae is the sample, relevant corresponding reference genomes would include at least other strains of the same species, other Saccharomyces species, and other yeast strains known to have either a relatively close evolutionary relationship with Saccharomyces yeasts or regions of relatively homologous genomic sequence.

A “normally haploid organism” is an organism which, during some substantial portion (e.g., hours, days, or years) of its lifespan, has a genome that is characterized by the presence of only a single copy of each chromosome of the organisms genome. By way of example, bacteria have only a single chromosome, and most bacteria are haploid except when replicating their genome prior to cell division.

The colloquial meaning of the term “likelihood” (i.e., the relative chance that an event or condition does or does not occur) is used in this disclosure, rather than any particularized definition that that word may have in the field of statistics or in certain statistical software packages.

The nucleotide sequence of a genome is “substantially known” if the nucleotide of at least 80%, and preferably at least 90%, of the genome has been determined

### DETAILED DESCRIPTION

Prior to development of the technology described herein, detection of polymorphisms in the genome of an organism required knowledge of the location of the polymorphism (and interrogation of that location) or sequencing of a portion of the genome containing the polymorphic form. Detection of unknown polymorphisms has therefore been difficult, time-consuming, and expensive. The methods described herein enable a skilled artisan in this field to rapidly and easily identify the location of known or unknown polymorphisms at a resolution of one or a few nucleotide residues. Using the technology described herein, it is now feasible to compare genomes (and portions of genomes) from individual organisms of the same or similar species in a single hybridization procedure (or a small number of such procedures) without the need to perform traditional DNA sequencing methods.

Overview

The methods described herein are based on the recognition that the intensity with which a polynucleotide probe hybridizes with a DNA strand having a complementary sequence is strongly dependent on both the presence or absence of a mismatched base pair within the complementary sequence and the location of such a mismatch. In general, for polynucleotide probes having a length of 10 to 60 nucleotide residues, a probe will exhibit a significantly lower hybridization intensity upon binding with a DNA molecule having a single mismatched nucleotide residue than it does upon binding with a DNA molecule having a completely complementary sequence (i.e., one with no mismatched nucleotide residues). Mismatches near the ends of the polynucleotide (i.e., within 5 nucleotide residues from the end of the probe) tend to lower hybridization intensity less than do mismatches nearer the center of the probe. Importantly, the decrease in hybridization intensity can be correlated with its distance from the ends of the probe, especially when the sequence context in which the mismatch occurs (e.g., the G-C content of the probe and the identity of the nucleotide residues flanking the mismatch) are taken into account. Although probes having lengths in the range from fewer than 10 (e.g., 7, 8, or 9) to about 60 (e.g., 65) nucleotide residues are operable, the probes preferably have lengths in the range from 25 to 45 nucleotide residues or within the narrower range from 25 to 35 nucleotide residues.

When a series of overlapping probes are hybridized with a DNA molecule in which a mismatch (relative to the sequence on which the probes are based) occurs, analysis of the relative hybridization intensities of the overlapping probes can pinpoint the location of the mismatch. The hybridization intensities of probes having the mismatch nearer their ends will be less affected by the mismatch than the hybridization intensities of probes more nearly centered over the mismatch. This observation can be applied in at least two ways. First, a series of overlapping probes can be used to assess the likelihood that a polymorphism occurs at a selected location in a genome (i.e., a location which the probes overlap). Second, by assessing hybridization intensity of probes which overlap at multiple locations in a genome (e.g., along a series of hundreds or thousands of base pairs), the locations of multiple (or multi-nucleotide residue) mismatches can be identified.

“Tiled” arrays of nucleotide sequences (i.e., arrays in which particular nucleotide sequences are attached to a substrate at discrete, known locations) are well known in the art. Many such arrays are commercially available, including arrays of entire yeast genomes, as described in the examples of this disclosure. This disclosure identifies how such arrays can be used to identify regions of a genome, or a portion of a genome, that are polymorphic with respect to the sequences represented in such arrays. Using such methods, genomic regions that differ from genomic sequences represented in an array can be rapidly and easily identified. Because such regions are of clear importance for a variety of purposes (e.g., identifying mutations; assessing genetic differences among strains and species), the methods disclosed herein represent an important advance in this field.

The Reference Genome

One important embodiment of the methods described herein relies on the existence of a genome having portions of known sequence. Preferably, substantially the entire sequence of the genome is known. With knowledge of its sequence, the reference genome can be used to generate probes corresponding to a portion (or the entirety) thereof. It is known that arrays of genomic probes having a known relationship to one another can be manufactured. By way of example, arrays of polynucleotide probes of fixed length (e.g., 25 nucleotide residues) and having a known overlapping relationship (e.g., 25-mer probes representing genomic regions serially shifted 5 nucleotide residues relative to one another, resulting in a 20-residue overlap between adjacent probes) have been described and made by others and are commercially available (e.g., the Affymetrix YTM arrays described in the examples of this disclosure).

Design and production of arrays that include overlapping probes of entire genomes or portions of them are known in the art and can be made with substantially no experimentation upon sequencing of any genome.

Sample Genome Polynucleotides

In the methods described herein, fragments of a sample genome are hybridized with overlapping probes of known sequence, and mismatches between the hybridized genomic fragments and the probes are detected.

The identity of the sample genome is not critical. Nonetheless, the utility of the methods is maximized when the genomic fragments are expected to bear some sequence similarity to at least a subset of the probes used. Preferably, genomic fragments are obtained from the same species (or a closely genetically-related species) as that from which the probe sequences are derived. As indicated by the results presented in the examples herein, the methods described in this disclosure can be used to distinguish genomic sequence differences among-strains of a single species and sequence differences arising from spontaneous mutations in a cultured strain.

It is immaterial whether the genomic fragments used the methods described herein are derived directly from fragmentation of genomic DNA, by amplification of one or more portions of genomic DNA, or are generated in another manner. Similarly, the methods used to generate the genomic fragments are not critical. Substantially any method that produces polynucleotides that are representative of the genomic region of interest and that are able to hybridize with the probes can be used. By way of example, DNase digestion of genomic DNA obtained from disrupted whole cells can be used, as described in the examples. Other methods of generating substantially random fragments can also be used. Preferably, the genomic fragments are similar in size. Genomic fragments having lengths from fewer than 10 (e.g., 7, 8, or 9) to about 60 (e.g., 65) nucleotide residues are useful in the methods described herein. Fragments having lengths in the range from 25 to 45 (preferably from 25 to 35) nucleotide residues are considered more likely to provide useful results than are longer or shorter fragments.

The methods described herein have been exemplified using genomic fragments derived from haploid organisms. The methods can also be used with genomic fragments derived from polyploid (e.g., diploid) organisms, from polyploid organism cells that are at a haploid stage in their life cycle (e.g., mammalian spermatozoa or ova), or from genomic sequences that occur in only one form in cells (e.g., X and Y chromosomes of men and organellar genomes such as those of mitochondria and chloroplasts). Other examples of suitable sources of genomic DNA for use in the methods describe herein are single-celled haploid organisms, such as bacteria, archaebacteria, and many yeast, multicellular organisms having haploid life stages, such as certain algae and fungi, and organisms having homozygous diploid genomes. Although the signal-to-noise ratio may be higher when genomic fragments are obtained from polyploid cells than when the fragments are obtained from haploid cells (or from viruses), the methods can nonetheless be used to analyze genomes of polyploid organisms, including humans and other animals.

Assessing the Likelihood that a Polymorphism Occurs at a Selected Nucleotide Position

In an important embodiment, the technology described herein relates to a method of assessing the likelihood that a polymorphism occurs at a selected nucleotide position in a first genome, relative to the same nucleotide position in a corresponding reference genome.

In order to make this assessment, fragments of the first genome are contacted with a plurality of polynucleotide probes under hybridizing conditions. Under such conditions, fragments that have a portion identical in sequence with that of a probe will hybridize with the probe. Furthermore, fragments that have a portion with a sequence that differs from the sequence of a probe by only a single mismatched nucleotide residue will also hybridize with the probe. Significantly, the hybridization intensity of the mismatched, fragment-probe pair will be measurably less than the hybridization of a perfectly matched fragment-probe pair. By assessing the intensity of hybridization of fragment-probe pairs, mismatched pairs can be identified by their lowered hybridization intensity.

Substantially any method of assessing hybridization intensity can be used. By way of example, the probes can be fixed to a support and the genomic fragments can be end-labeled with a fluorescent moiety, permitting hybridization of the fragments with the support-linked probes and subsequent detection of hybridized fragments following hybridization and washing of the probes and fragments. Similarly, chromogenic or radioactive labels can be used in place of fluorophores. The assessment of hybridization intensity need not employ any particular detectable label. Any method known in the art or hereafter developed for assessing hybridization of the probes and fragments can be used.

In the method, probes are selected so that they are completely complementary to different, but overlapping, portions of the reference genome that include the selected nucleotide position. By comparing the hybridization intensities of probes that overlap the selected nucleotide position, occurrence of a polymorphism (relative to the reference genome sequence) can be identified because probes that are more nearly centered over the selected nucleotide position will exhibit a greater decrease in hybridization intensity than probes that overlap the selected nucleotide position nearer their ends.

If probes that serially overlapped one another by single-nucleotide offsets were used, this gross observation of hybridization intensity might be sufficient to pinpoint the location of a polymorphism. However, such a probe set includes a relatively large number of probes. Use of statistical methods permits a smaller number of probes, offset from one another by a larger number of nucleotide residues (e.g., 2, 3, 4, 5, 6, 7, or more), to yield similarly precise results. It is important (for statistical purposes) that the probes overlap sufficiently that assessments of hybridization can be made for at least two probes with a genomic nucleotide residue of interest. For that reason, the offset of probes used to assess occurrence of polymorphisms in a region of genomic DNA should not exceed one-half the length of the probes used, and preferably does not exceed about one-third that length. Viewed another way, it is preferable that assessments of the likelihood that a polymorphism occurs at a selected genomic nucleotide residue should be based on assessments of hybridization intensity for at least two, and preferably three, four, or five or more probes that overlap that nucleotide residue.

Particularly where large genomic regions are to be assessed using these methods, a decrease in the number of probes required simplifies the methods, permits more efficient use of reagents and equipment, and reduces the need for multiple or very large probe arrays. Increasing the offset between probes can also decrease the number of probes in the array that correspond to the selected nucleotide residue. Preferably, hybridization intensity values are assessed for at least a few probe-fragment pairs. As described in the examples herein, an offset value of 5 has been found operable.

The expected hybridization intensity can be calculated for each probe used in the methods, based on the presumed occurrence of either a complementary base or a non-complementary base at the nucleotide residue corresponding to the nucleotide position. By comparing the predicted hybridization intensity for the probe with the hybridization intensity measured experimentally, one can estimate the likelihood that a polymorphism occurs at the selected nucleotide residue. By combining the estimates for each of the probes that overlaps the selected nucleotide residue, a more reliable estimate of the likelihood of polymorphism occurrence at the selected nucleotide residue can be generated.

In an important embodiment, both fragments of the sample genome and fragments of a reference genome are hybridized with the same set of reference genome probes (e.g., a single probe array). The sample genome fragments and the reference genome fragments are differentially labeled (e.g., each can be labeled with a spectrally distinguishable fluorophore). In this embodiment, the hybridization intensity corresponding to individual sample genome fragments can be directly compared (i.e., using precisely the same set of reference probes and hybridization conditions) with the hybridization intensity corresponding to fragments of the reference genome, eliminating the need to normalize results obtained by separately hybridizing sample genome fragments with a first set of reference probes (e.g., with a first probe array) and hybridizing reference genome fragments with a second set of the same reference probes (e.g., with a second, identical probe array). Similarly, multiple copies of sample genome fragments, multiple copies of reference genome fragments, or, both can be hybridized with a single set of reference genome probes (e.g., a single tiled array of reference probes linked to a support). Each set of fragments should be distinguishably labeled. In this embodiment, the quantities of fragments used should be controlled so as to avoid saturating the probe set (i.e., to avoid competitive inhibition among fragments from different sources).

Assessing the Location of a Polymorphism in a Genome

The methods described herein can be used, as described above, to predict the likelihood that a polymorphism occurs at a particular genomic location. By performing that prediction at multiple locations within a genome, the location of a polymorphism can be predicted. By way of example, the likelihood that a polymorphism occurs can be assessed at each of a plurality of closely-spaced nucleotide positions in the first genome. A polymorphism is predicted to occur at the position having the highest estimated likelihood that a polymorphism occurs at or near that position.

Similarly, the likelihood that a polymorphism occurs can be assessed at each of a plurality of consecutive nucleotide positions (e.g., 10, 50, 100, or 1000 or more consecutive nucleotide residues) in the first genome, or for substantially every nucleotide residue in the genome. A polymorphism is predicted to occur at positions having relatively high estimated likelihoods, and a polymorphism predicted within a region can be localized within that region to the nucleotide residue exhibiting the highest local likelihood (i.e., the highest likelihood within the length of the probes used). Single nucleotide polymorphisms are expected to yield predictions of increased polymorphism likelihood over a span of a few to several nucleotide residues. (the resolution depending on the number of overlapping probes and their offset). Longer polymorphisms (e.g., insertions and multi-nucleotide residue sequence differences) and closely-spaced (i.e., within the span of the probe length used) single-nucleotide polymorphisms are readily detected but expected to yield less specific predictions of their locations.

Methods of predicting the hybridization intensity for a probe should preferably take into account factors that can foreseeably and significantly affect that value. By way of example, hybridization intensity values should be predicted based both on the occurrence of a non-complementary nucleotide residue at the selected nucleotide position and on the occurrence of a complementary nucleotide residue at selected nucleotide position. Furthermore, the observed variance in hybridization intensity measurements (e.g., the variance observed when hybridizations are performed using fragments of the reference genome) should also be taken into account. The G-C content of the probes can also affect hybridization intensity, and its effect should be accounted for, as should the cross-hybridization potential of the probe, and the tendency of the probe to form secondary structure that impedes hybridization.

In one method for predicting the likelihood that a polymorphism occurs at a selected nucleotide position, a prediction signal (Lₖ) value is calculated from the predicted and assessed hybridization intensity values obtained for each of i probes that overlap the selected position. By way of example, a suitable Lₖ value can be calculated from the following formula (1).

$${L}_{k} = ( {log}_{10} e ) {\sum }_{i} \frac{{( {x}_{i} - {μ}_{ni} )}^{2} - {( {x}_{i} - {μ}_{pi} )}^{2}}{2 {σ}_{i}^{2}} \qquad ( 1 )$$

In that formula, xᵢ is the assessed hybridization intensity for an individual probe, μₙᵢ is the predicted hybridization intensity for the individual probe when no polymorphism occurs at the nucleotide position, μₚᵢ is the predicted hybridization intensity for the individual probe when a polymorphism occurs at the nucleotide position, and σᵢ is the intensity variance for the probe.

A positive value of Lₖ indicates that a polymorphism likely occurs at the nucleotide position, and a negative value of Lₖ indicates that a polymorphism likely does not occur at the nucleotide position. More positive values of Lₖ (i.e., larger positive numbers) indicate increased likelihood of polymorphism occurrence, and more negative values (i.e., larger negative numbers) indicate decreased likelihood of polymorphism occurrence. μₙᵢ values can be assessed based on hybridization intensity values observed when fragments of the reference genome are hybridized with the probe, for example. σᵢ values can be assessed based on the variance in hybridization intensity values observed when fragments of the reference genome are hybridized with the probe. Values for μₚᵢ is calculated for each of the i probes using the formula μₚᵢ=μₙᵢ−Dᵢ, wherein Dᵢ is calculated according to formula (2).

Dᵢ=α+β(GCᵢ)+γ(PMᵢ−MMᵢ)+δPMᵢ (2)

In formula (2), Dᵢ is the signal difference for a completely complementary probe overlapping a polymorphism, GCᵢ is the G-C content of the probe, PMᵢ is the hybridization intensity of the probe when hybridized with a completely complementary polynucleotide, and MMᵢ is the hybridization intensity of the probe when hybridized with a complementary polynucleotide having a single mismatched base at its center (e.g., the mismatched probes in the Affymetrix YTM arrays described in the examples). Each of α, β, γ, and δ is a constant derived from statistical fitting of data obtained from hybridization of the probe with fragments of the reference genome. The identity of the statistical package used to fit such data is not critical.

Kits

Also disclosed herein are kits useful for assessing occurrence of one or more polymorphisms in a first genome, relative to a corresponding reference genome. Such a kit includes i) an array comprising a plurality of polynucleotide probes attached at discrete known locations on a support, wherein the probes are completely complementary to different, but overlapping, portions of the reference genome; and ii) a digital storage medium including an algorithm for comparing assessed hybridization intensity values for the probes with predicted hybridization intensity values, whereby occurrence of one or more polymorphisms in the first genome can be assessed. It is not critical that the array and the digital storage medium be packaged or shipped together. They may be shipped, packaged, and used at discrete locations by the same individual or by multiple persons practicing the methods described herein.

### EXAMPLES

The subject matter of this disclosure is now described with reference to the following Examples. These Examples are provided for the purpose of illustration only, and the subject matter is not limited to these Examples, but rather encompasses all variations which are evident as a result of the teaching provided herein.

On the basis of a single experimental hybridizations, we were able to accurately detect the single nucleotide changes that distinguish two genomes. Recently a microarray design superficially similar to that described herein was used as a preliminary screen to identify possible mutations in the pathogen H. pylori (Albert et al., 2005, Nat. Methods 2:951). In that method, the initial screen was followed by manufacture of targeted resequencing microarrays. By contrast, the methods described herein rely on only a single experiment to derive a statistical measure of the likelihood that a polymorphism occurs at a particular site. The methods described herein are optimal when direct comparisons are made to the reference strain represented on the microarray. However, these methods can also be used to compare two non-reference genomes and identify the SNPs that distinguish them with only minimal added cost in terms of false negatives and false positives. Although the algorithms described herein are trained on a set of known base substitutions, the algorithms also detected single base deletions and insertions as well as large deletions with near-nucleotide accuracy in the prediction of breakpoints. Any genomic variation that results in novel sequence, such as inversions or retrotransposon-insertions should be detected using the SNPscanner algorithm.

The simplicity and affordability of the methods described herein enable individual laboratory groups to devise and employ new and truly comprehensive genomic approaches to mendelian and complex genetics and the characterization of mutants obtained through genetic and suppressor screens. In addition, complete knowledge of nucleotide-diversity facilitates answering questions regarding the mutagenic effect of phenomena such as aging and recombination on a genome-wide scale. By representing entire genomes of organisms other than yeast on oligonucleotide microarrays using a similar redundant design, the method described herein can be used to analyze the genomes of such other organisms. Although increased genome complexity can increase the amount of noise in the signal obtained using the methods described herein, genome-wide prediction of all sequence variants is possible in genomes larger than the yeast genome, including important model organisms such as Arabidopsis, C. elegans, and D. melanogaster.

In studies described in this example, a commercially available (from Affymetrix) high-density yeast tiling microarrays (YTM) with overlapping 25-mers spaced an average of base pairs (bp) apart was used to provide complete and approximately 5-fold redundant coverage of the entire Saccharomyces cerevisiae genome. The 5 to 7 measurements of a nucleotide's effect on hybridization efficiency provided by this design were used to predict the presence and location of SNPs and deletion breakpoints throughout the entire yeast genome.

The YTM that was used has approximately 2.6 million perfect match (PM) probes and approximately 2.6 million corresponding mismatch (MM) probes. We modeled the decrease in PM probe intensity caused by a single SNP as a function of the SNP's position within the probe, the probe's G-C content, the nucleotide sequence surrounding the SNP, and the hybridization intensity obtained using a non-polymorphic reference (S288C) genome (strain FY3). In order to determine the parameters of the model that would best approximate the observable data, we used hybridization data obtained with a training set of nearly 25,000 high-quality SNPs in strain RM11-1a, all of which had been identified by direct comparison of the genomic sequences.

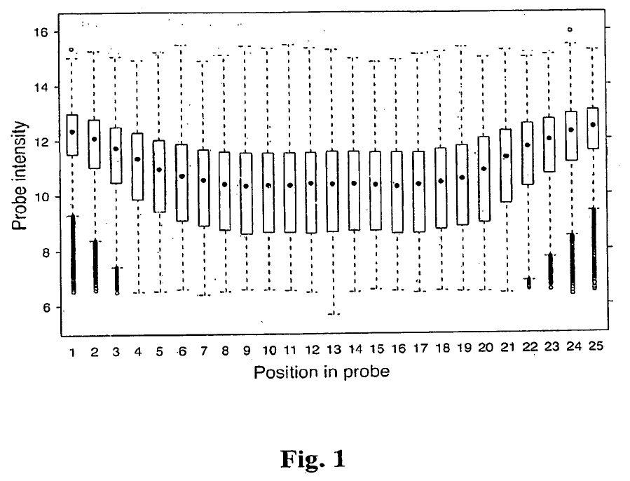

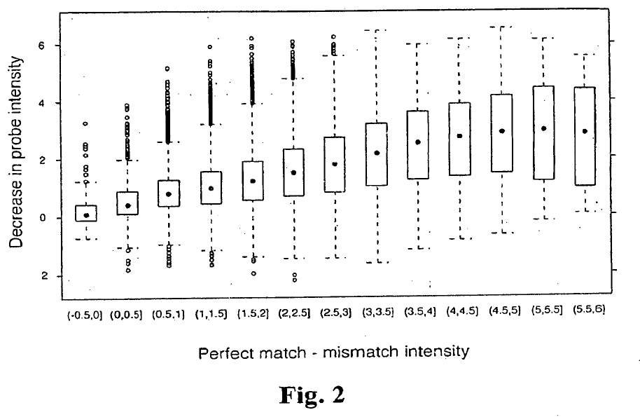

The model prepared as disclosed herein predicts the intensity of a probe in the presence of a specified SNP (FIG. 1, FIG. 2), and is employed in the algorithm disclosed herein (designated “SNPscanner”), which calculates the log of the likelihood ratio (the “prediction signal”) for the occurrence of a SNP at each nucleotide in the genome using all probes that interrogate that nucleotide. By scanning the entire yeast genome, we identified SNPs by observing regions of elevated signal, in which the position of the peak value is the predicted polymorphic site.

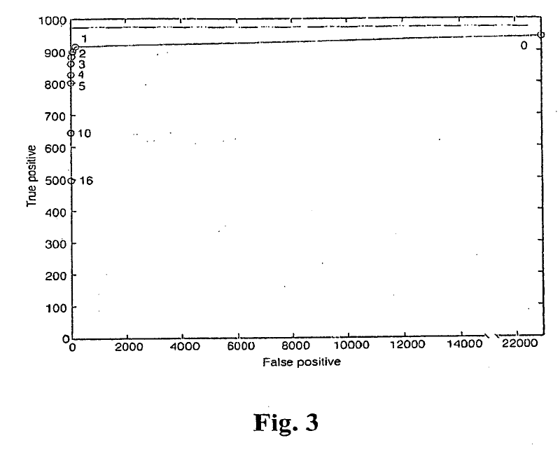

We tested the performance of SNPscanner using a set of 981 high quality SNPs from yeast strain RM11-1a that were not included in the training set. We assessed the false positive rate by using SNPscanner to predict SNPs from an independent hybridization of the reference strain, where no true polymorphisms are expected. At a prediction signal threshold of 1 we detected 915 (93.3%) known SNPs in RM11-1a and called 177 false positives in the reference strain (FIG. 3). By increasing the prediction signal threshold to 5 and applying a heuristic filter, we eliminated all false positives and retained 760 (77.5%) of real SNPs. Analysis of this set of correctly predicted SNPs showed the sequence confirmed SNP to be within two base pairs of the predicted site 87.1% of the time.

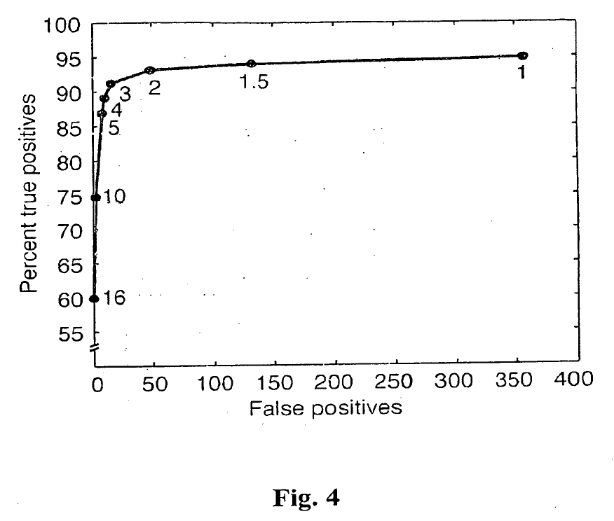

To test our ability to predict a large number of SNPs, we analyzed the sequenced, but highly diverged yeast strain designated YJM789, which was originally recovered from an AIDS patient (Gu et al., 2005, Proc. Natl. Acad. Sci. USA 102:1092). We selected a set of 30,303 sequence-confirmed SNPs that were separated from each other by at least 25 bp and represented on the YTM. Analysis of a single hybridization using the SNPscanner algorithm yielded 28,737 (94.8%) correctly predicted SNPs at a prediction signal threshold of 1 (FIG. 4). At a prediction signal threshold of 5, we detected 86.9% of known SNPs and only called 7 false positives in a similar analysis of the reference genome. Those false positives were readily excluded by our heuristic filter.

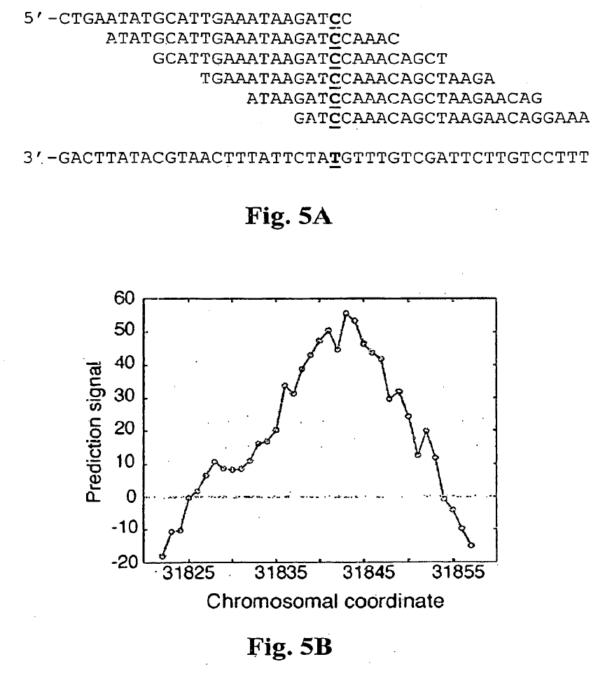

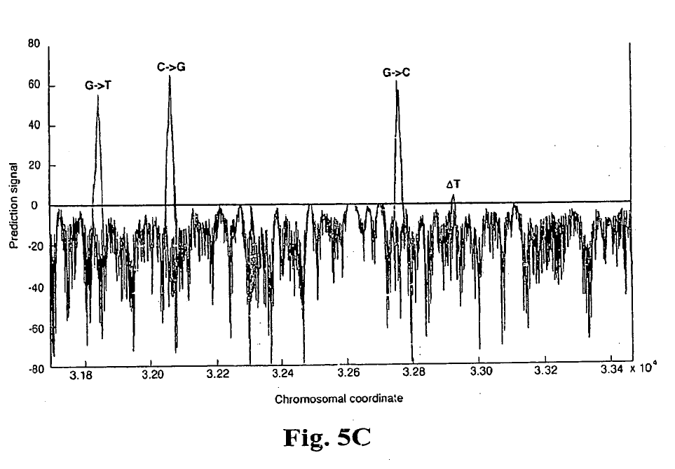

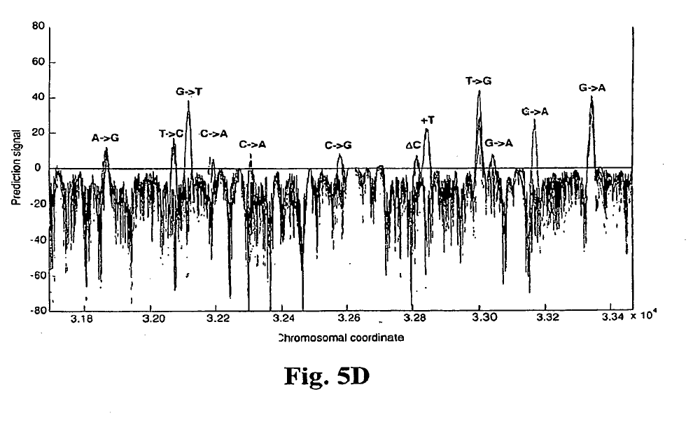

To test our ability to accurately detect a very small number of sequence differences that distinguish two genomes, we analyzed spontaneous mutants in the yeast strain FY3. Independent clones from the same archival isolate were grown up, and mutants in the CAN1, GAP1 or FCY1 genes were selected on plates containing canavanine sulfate, D-serine and D-histidine, or 5-fluorocytosine, respectively. For each mutant, we hybridized total genomic DNA with a single YTM and analyzed the data using the SNPscanner algorithm (FIGS. 5A and 5B). In each of four can1 mutants, we detected a single signal at the CAN1 locus that fulfilled our prediction criteria for a SNP (FIG. 5C). Amplification and sequencing of the CAN1 locus identified a single base substitution in each of three mutants (31844G→T; 32064 C→G; 32757 G→C) and deletion of a single thymine residue in a run of four thymine residues in the fourth (32924ΔT). Although the prediction signal for this thymine deletion was comparatively low, its detection is notable, because no insertions or deletions (indels) were included in the set of SNPs used to train the model.

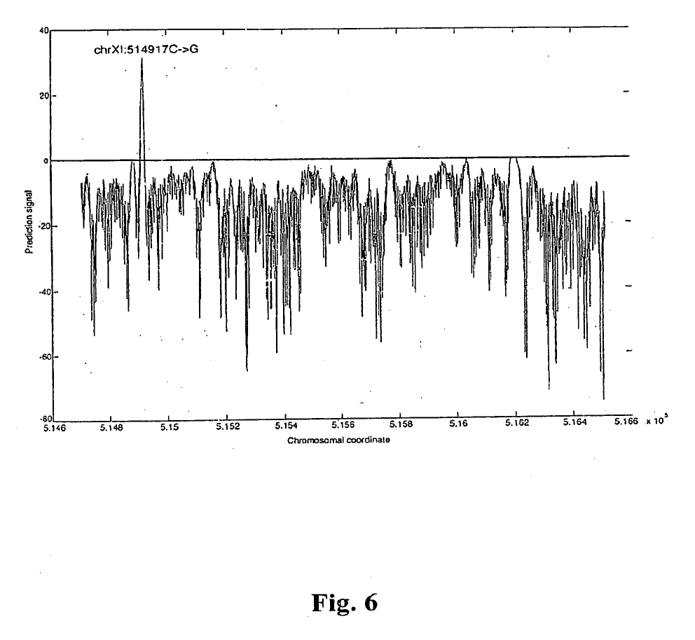

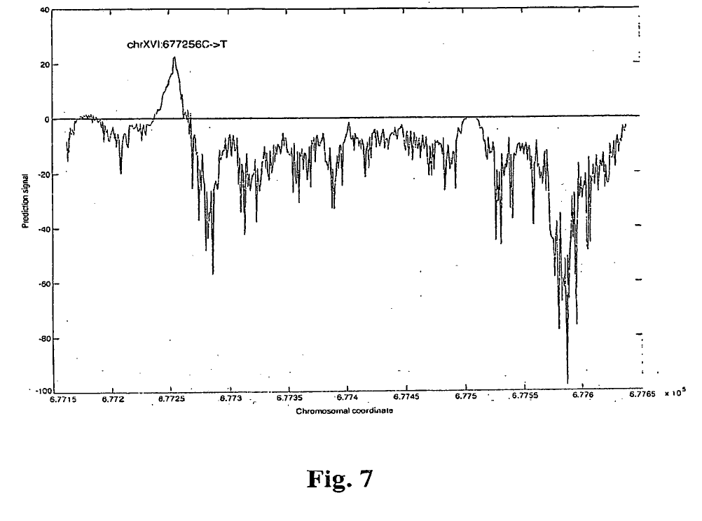

Analysis of DNA from a yeast mutant strain that is resistant to D-histidine/D-serine using SNPscanner algorithm predicted a mutation in GAP1 (chromosome XI), which we confirmed as a 514919 C->G substitution by sequence analysis (FIG. 6). Similarly, the SNPscanner algorithm accurately predicted a mutation in FCY1 (chromosome XVI) for a yeast mutant strain that is resistant to 5-fluorocytosine (677256 C->T; FIG. 7). These results demonstrate that the SNPscanner algorithm can be used to detect a variety of single base changes, including a single-base deletion, at several different loci in the genome and map them to within 2 bp of the verified substitution (data shown in Table 1).

TABLE 1
SNPs predicted in spontaneous mutants in the reference genome background.

| | | | | | Predictions | |
|---|---|---|---|---|---|---|
| | | | | | excluding | |
| | | | | | repetitive | Number of |
| | SNPscanner prediction | | | Rank of | DNA features | sequence |
| | at expected locus | Confirmed | No. of | expected | and array | confirmed |
| Mutant | (prediction signal) | Mutation | predictions | mutation | artifacts | mutations** |
| Can1-1001 | chrV: 32,758 (62) | chrV: 32,758G→C | 75 | 1 | 5 | 3 |
| Can1-1002 | chrV: 32,929 (4.5) | chrV: 32,924ΔT*** | 294* | — | 5 | 2 |
| Can1-1003 | chrV: 31,844 (55.6) | chrV: 31,844G→A | 12 | 1 | 3 | 3 |
| Can1-1004 | chrV: 32066 (65) | chrV: 32,064C→G | 69 | 1 | 4 | 3 |
| Gap1-1002 | chrXI: 514,919 (105) | chrXI: 514,919C→G | 22 | 1 | 4 | 2 |
| Fcy1-1001 | chrXVI: 677,256 (178) | chrXVI: 677,256C→T | 414 | 5 | 120**** | 2 |

*filtering SNPscanner predictions resulted in exclusion of the expected mutation in CAN1 for this sample
**a single mutation was predicted in all samples, chrIV: 548350 and confirmed as a chrIV: 548348T→C mutation. Additional mutations were sequence-confirmed in 3 spontaneous mutants.
***the deletion occurs within a run of 4 T's from coordinate 32,927-32,924 and therefore cannot by localized to the precise nucleotide
****the high number of predictions appears to result from a microarray manufacturing defect that results in numerous discrete foci of decreased intensity scattered across the microarray

TABLE 2
SNPs predicted in CEN.PK CAN^{R} mutants

| | SNPscanner | | | Predictions in | |
|---|---|---|---|---|---|
| | prediction at CAN1 | Confirmed | Number of predictions | non-repetitive | Rank of expected |
| Sample | (prediction signal) | mutation | genome-wide | DNA | mutation in CAN1 |
| Can1-2003 | chrV: 32,486 (0.66) | chrV: 32,487G→T | 12* | 0 | — |
| Can1-2004 | chrV: 32,119 (38.5) | chrV: 32,119G→T | 32 | 16 | 1 |
| Can1-2005 | chrV: 33,168 (28.1) | chrV: 33,169G→A | 21 | 4 | 2 |
| Can1-2007 | chrV: 32,579 (8.4) | chrV: 32,580C→G | 33 | 5 | 1 |
| Can1-2008 | chrV: 32,308 (8.2) | chrV: 32,304C→A | 3* | 3 | — |
| Can1-2009 | chrV: 32,814 (7.3) | chrV: 32811ΔC*** | 5 | 5 | 5 |
| Can1-2010 | chrV: 32,072 (17.3) | chrV: 32,077T→C | 47* | 15 | — |
| Can1-2011 | chrV: 31,195 (13.6) | chrV: 32195C→A | 5 | 2 | 1 |
| Can1-2013 | chrV: 33040 (8.2) | chrV: 33043G→A | 74 | 56 | 10 |
| Can1-2014 | chrV: 32843 (22.9) | chrV: 32842 + T**** | 583 | 556***** | 3 |

*filtering scanner predictions according to our criteria resulted in exclusion of the expected mutation in CAN1.
***the deletion of a single C occurs within a run of three Cs from coordinates 32,813-32,811 and therefore cannot be localized to the precise nucleotide
****the insertion of a single T occurs within a run of 6 Ts from coordinates 32,847-32,842 and therefore cannot be localized to the precise nucleotide
*****the high number of predictions appears to result from a microarray manufacturing defect that results in numerous discrete foci of decreased intensity scattered across the microarray

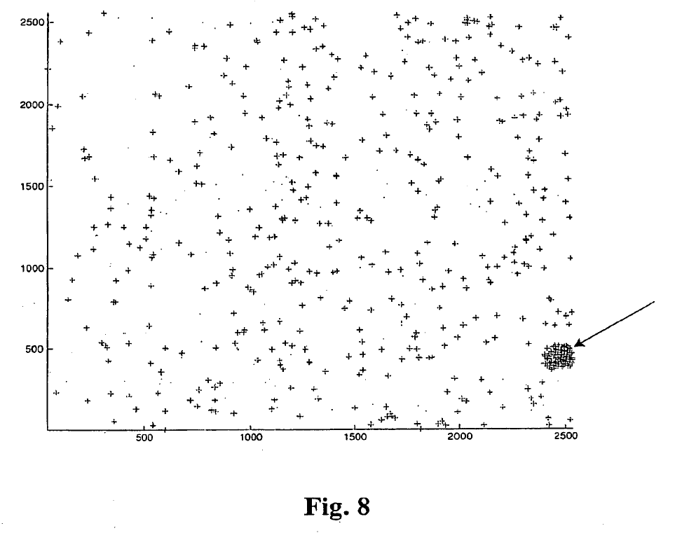

In addition to the anticipated mutations, the SNPscanner algorithm yielded 12 to 414 additional predictions per genome (Table 1). We identified two main causes of experimental noise: false positives that fell within repetitive genomic features, such as retrotransposons and telomeres, which we subsequently excluded (Table 1), and manufacturing defects in microarrays, which we removed computationally (FIG. 8). We ranked the remaining predictions based on signal strength for each mutant and found the expected mutation in the top five predictions for all mutants except the one resulting from an indel (can1-1002). One SNP prediction (chr IV: 548,350, sequence confirmed as 548348G->C) was common to all samples, suggesting an early mutation event that preceded subsequent experiments (for example, during single colony purification from the archived stock culture). Using sequence confirmation of high quality predictions passing our filtering criteria, we identified additional unique mutations in three of the six spontaneous mutants (Table 1). Thus, the SNPscanner algorithm is sufficiently sensitive to detect a small number of base changes that distinguish two genomes with no a priori knowledge of the variants' location. These results indicate that there were only a small number of mutations (<5) associated with the generation of spontaneous drug mutants used in these studies.

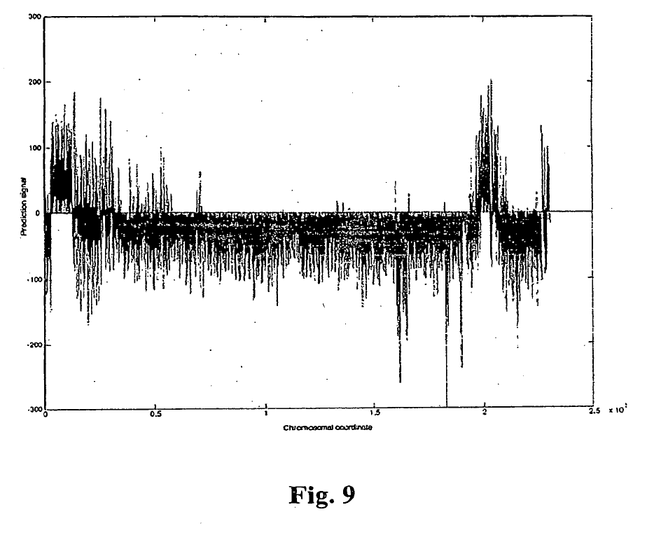

We extended our approach to characterize the genome of the non-sequenced laboratory yeast strain designated CEN.PK, which is commonly used in continuous culture experiments. CEN.PK shares ancestry with the reference strain, S288C, but some genes are known to be absent in CEN.PK (Daran-Lapujade et al., 2003, FEMS Yeast Res. 4:259). We obtained a nucleotide-resolution comparison with the reference sequence by analyzing data from a single hybridization of CEN.PK DNA using the SNPscanner algorithm. The results indicate that CEN.PK has a strikingly mosaic structure, with large portions of the genome sharing essentially complete sequence identity with FY3 interspersed with regions of sequence divergence and large deletions (FIG. 9).

The ability of the SNPscanner algorithm to detect single mutations on a genome-wide scale in a non-reference genome was analyzed. This was expected to be a more difficult statistical problem. We selected ten spontaneous Can^{R} mutants in the CEN.PK strain background and hybridized genomic DNA to the YTM. SNPscanner predictions correctly identified a mutation in nine of the ten mutants, as well as three polymorphic sites present in the wild type CEN.PK background (FIG. 5D). We confirmed the sequence of all CAN1 mutations and polymorphisms in the ten Can^{R} mutants. Seven of the nine detected mutants had base substitutions in CAN1, one contained a 1 bp insertion and another had a 1 bp deletion. All mutations were confirmed as lying within 7 bp of the predicted site (Table 2).

SNPscanner prediction signals were highly reproducible across multiple experiments. Genome-wide SNP predictions for each CEN.PK can1 mutant were compared to SNP predictions for CEN.PK wild-type DNA using our heuristic filter. This comparison resulted in prediction of fewer than 100 SNPs genome-wide that are not predicted to exist in wild-type CEN.PK for nine of the ten can1 mutants (Table 3). In most instances, excluding predictions that occurred in repetitive regions further reduced the total number. Using that approach, the identified can1 mutation was retained for seven of nine mutants. We ranked the remaining predictions and observed that the sequence-confirmed mutation was in the top ten predictions for all seven of those mutants. So even in this more challenging statistical situation, the SNPscanner algorithm successfully detected most of the single nucleotide sequence differences and mapped their location to within a few nucleotides. Mutations predicted in our collection of CEN.PK and FY3 spontaneous mutants correspond to nine of the twelve possible base substitutions that result in six of the eight possible mismatches between probe and sample. Thus, the SNPscanner algorithm can detect single base-pair indels, in addition to virtually all base substitutions.

We sought to apply our genome-wide mutation detection approach to biological questions that had remained refractory to traditional genetic analytical techniques. Table 3 illustrates how genome-wide mutation mapping facilitates a genomic approach to genetics. Following SNPscanner analysis of a defined approximately 50 kb critical region, we identified mutations in AEP3 that segregated with the ability and inability to use acetate as a carbon source. We complemented a positional cloning project to predict and confirm mutations in AEP3 (a peripheral mitochondrial inner membrane protein) that are causative of a growth defect on a non-fermentable carbon source (Table 3; Ellis et al., 2004, J. Biol. Chem. 279:15728).

TABLE 3

| | | SEQ | |
|---|---|---|---|
| | | ID | |
| Strain | Phenotype | NO: | Sequence in AEP3 |
| FY3 | Acetate+ | 8 | . . . TTCAAATGGGTGAACA . . . |
| CP1AB | Acetate− | 9 | . . . TTCCAATGGGTGAACA . . . |
| E2.1A | Acetate+ | 10 | . . . TTACAATGGGTGTGCA . . . |
| E2.1C | Acetate− | 11 | . . . TTCCAATGGGTGAACA . . . |
| E3.4B | Acetate+ | 12 | . . . TTCAAATGGGTGAACA . . . |
| E3.4A | Acetate− | 13 | . . . TTCCAATGGGTGAACA . . . |

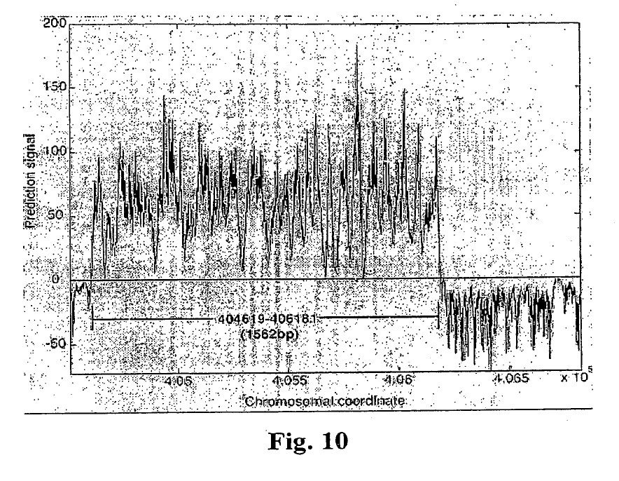

We also used the methods described herein to determine the genetic basis of an unusual phenotype. Deletion of AMN1 results in up-regulation of daughter specific genes and a non-clumpy growth phenotype (Yvert et al., 2003, Nat. Genet. 35:57). However, when we deleted AMN1 in an S288C-like strain (strain BY4716), we recovered a transformant that displayed low expression of daughter specific genes and a clumpy phenotype (strain YEF1695). Deletion of AMN1 in YEF1695 was confirmed by sequence analysis. Independent deletions of AMN1 in both BY4716 and RM11-1a yielded the expected phenotype, suggesting the presence of a suppressor mutation in YEF1695: Preliminary genetic analysis tended to indicate the presence of an unlinked suppressor mutation. We hybridized genomic DNA from YEF1695 to the YTM. Analysis using the SNPscanner algorithm confirmed the deletion of AMN1 and identified an additional deletion on chromosome XII (FIG. 10A). The predicted deletion spans approximately 1.5 kb and includes the majority of the coding region of ACE2. Subsequent sequence analysis confirmed the predicted breakpoint were within 1 bp of confirmed sites (FIG. 10).

The deletion of ACE2 provides a plausible explanation for both aspects of the aberrant phenotype of YEF1695. ACE2 encodes a transcription factor, which is thought to drive the transcription of genes with daughter-specific expression (Colman-Lerner et al., 2001, Cell 107:739). Its absence in YEF1695 probably causes a low expression of the daughter cell-specific genes, some of which are required for cell separation after budding (e.g. CTS1, which encodes chitinase). Moreover, deletion of ACE2 alone results in a clumpy phenotype (Racki et al., 2000, EMBO J. 19:4524), and clumpiness Segregates with the ACE2 locus in a cross between YEF1695 and RM11Δamn1.

Previous studies have indicated the occurrence of characteristic gene expression patterns and large-scale gene duplication and deletion in yeast strains that are experimentally evolved under a nutrient limiting condition (Ferea et al., 1999, Proc. Natl. Acad. Sci. USA 96:9721; Dunham et al., 2002, Proc. Natl. Acad. Sci. USA 99:16144). However, the extent and nature of nucleotide changes that occur during this process have remained completely unknown.

We sought to assess the degree of sequence variation that accumulated in a strain of yeast subjected to experimental evolution under sulfur limitation in continuous culture. We compared the signals obtained using the SNPscanner algorithm for DNA of the ancestral strain, CEN.PK, and for DNA of two clones from the same population that had undergone experimental evolution under sulfur limitation for 63 (strain DBY11130) and 123 (strain DBY11131) generations. We compared our set of predictions to those made for CEN.PK CAN^{R} mutants to exclude common predictions that were the result of systematic error. SNP predictions that fell within repetitive regions were considered to be unreliable and excluded from further analysis.

At a prediction signal >5 we called a small number of predicted SNPs in strains DBY11130 and DBY11131, twelve of which were common to the two strains (Table 4). We sequence-confirmed the presence of single strain-specific mutations in DBY11130 and DBY11131 (Table 4). The relatively small number of mutations strongly suggests that the events associated with adaptive evolution in chemostats do not involve even transient genome-wide mutagenesis, and is consistent with the experience that in yeast, evolved strains are rarely, if ever, found to have mutator phenotypes. This is in contrast to studies using E. coli grown in batch conditions, in which conditions mutator phenotypes have been observed in numerous independent cultures (Sniegowski et al., 1997, Nature 387:703). The small number of mutations identified in our experiments means that comprehensively identifying and experimentally verifying mutations that are important for adaptation during studies of experimental evolution will be feasible in the near future.

TABLE 4
Predicted SNPs detected in a yeast strain subjected
to experimental evolution

| | | Number of | Sequence |
|---|---|---|---|
| | Generations under | SNPscanner | confirmed |
| Strain | sulfur limitation | predictions | mutation |
| DBY11130 | 63 | 19 unique SNPs | ChrIV: 498,631: |
| | | | C->A in REG1 |
| | | | (D749Y) |
| DBY11131 | 123 | 6 unique SNPs | ChrVII: 858,403: |
| | | | G->C in TIM13 |
| | | | (A38P) |
| DBY11130 | — | 12 shared SNPs | — |
| and | | | |
| DBY11131 | | | |

The materials and methods used in the work presented in these examples are now described.

Yeast Strains and Media

For initial experiments the haploid S288C-derived yeast strain FY3 (MATa ura3-52) was used. The haploid strain designated CEN.PK (MATa), derived from strain CEN.PK122, was used for testing the ability to detect single mutations in a non-reference strain and for performing experimental evolution in the chemostat. Gene mapping was performed in the diploid strain designated CP1AB (MATa/α), which is closely related to S288C.

Selection of spontaneous mutants was performed on plates containing one of i) synthetic complete medium with 60 milligrams per liter canavanine sulfate and no arginine; ii) yeast nitrogen base without amino acids or ammonia, supplemented with uracil and 0.5% proline as a nitrogen source, 3 millimolar D-histidine, and 1.5 millimolar D-serine; and iii) yeast nitrogen base supplemented with uracil and a final concentration of 1 millimolar 5-fluorocytosine. All solid media contained 2% glucose. For spontaneous mutant selections, ten independent overnight cultures were inoculated from the same single colony purified clone. We plated approximately 5×10⁶ cells from each culture on independent plates and allowed two days growth at 30 degrees Celsius. One mutant from a single colony on each plate was picked and grown up on non-selective media.

Yeast strain BY4716 (MATα Lys2Δ0), which is an S288C-derived strain, was transformed with an AMN1 construct as described (Yvert et al., 2003, Nat. Genet. 35:57).

Experimental evolution of CEN.PK was performed in a defined medium as reported previously (Saldanha et al., 2004, Mol. Biol. Cell 15:4089) under sulfur limitation by using a concentration of 3 milligrams per liter of ammonium sulfate. Cultures were maintained in steady state growth by means of a chemostat as described (Saldanha et al., 2004, Mol. Biol. Cell 15.4089).

Yeast strains grown for the purpose of DNA preparation were grown in 100-milliliter cultures of yeast extract, peptone and 2% dextrose (YPD) medium.

DNA Isolation, Microarray Hybridization and Sequencing

Qiagen Genomic Tips and Genomic DNA Buffers were used, per the manufacturer's instructions to obtain high quality DNA. Three samples of 10 micrograms of purified DNA were digested to 25-50 bp fragments using 0.15 unit of DNaseI in the presence of 1.5 millimolar cobalt chloride and 1× One-Phor-All buffer for 5, 10 or 15 minutes at 37 degrees Celsius. DNaseI was heat inactivated at 100 degrees Celsius for 15 minutes. The digestion-products were analyzed on a 2% agarose gel and a completely digested sample was selected for labeling.

DNA was end-labeled using 25 units of terminal transferase and 1 nanomole of biotin-N6-dATP (Enzo Life Sciences) at 37 degrees Celsius for 1 hour. The reaction was stopped by the addition of 5 microliters of 0.2 molar EDTA. The labeled DNA was mixed with 0.150 micrograms of BSA, 30 micrograms of salmon sperm DNA, and 5 microliters of the 3 nanomolar B3 biotin control oligonucleotide (Affymetrix) in a hybridization buffer containing a final concentration of 100 millimolar MES salts (4:3 MES hydrate: MES sodium salt), 1 molar NaCl, 20 millimolar EDTA and 0.01% TWEEN®-20 100 to a total volume of 300 microliters.

The sample was heated to 99 degrees Celsius for 5 minutes, cooled to 45 degrees Celsius for 5 minutes, and spun in a microcentrifuge for 5 minutes to collect any particulate matter. 200 Microliters of the sample solution was applied to the Affymetrix Yeast Tiling (Reverse) Microarray (YTM), which had been pre-incubated with hybridization buffer for minutes at 45 degrees Celsius. The hybridization reaction was allowed to occur for 20 hours at 45 degrees Celsius at 60 rotations per minute in a hybridization oven.

Following hybridization, the reaction solution was removed and the array was washed using an Affymetrix GENECHIP FLUIDICS STATION™ 450 using the EukGe-wash4v2 protocol. The protocol entails a non-stringent wash A (0.9 molar NaCl, 60 millimolar NaH₂PO₄, 6 millimolar EDTA and 0.01% TWEEN®-20 detergent) followed by a stringent wash B (100 millimolar MES salts, 0.1 molar NaCl and 0.01% TWEEN®-20). The array was stained using 10 micrograms per milliliter R-phycoerythrin-streptavidin with 2 milligrams per milliliter BSA in 1× staining buffer (100 millimolar MES salts, 1 molar NaCl and 0.05% TWEEN®-20) in a volume of 600 microliters. Biotinylated anti-streptavidin antibody (3 micrograms per milliliter) with goat IgG (0.1 milligram per milliliter) and 1.2 milligrams of BSA in 1× stain buffer in a total volume of 600 microliters was added to amplify the signal. Finally, a second staining using the R-phycoerythrin-streptavidin mixture was performed.

The YTM was scanned once at 0.7 micrometer resolution using the Affymetrix scanner. The hybridization intensity of the 9 central pixels was determined (representing 75^{th} percentile of the entire feature) and an average intensity computed using the GeneChip Operating software to yield a “.CEL” file containing the average intensity at each feature.

All sequences were confirmed by polymerase chain reaction (PCR) amplifying the relevant and cycle sequencing using typical methods.

Analysis of Probe Behavior and Design of Algorithm

By contrast to previous reports using tiling arrays in which probes are evenly spaced across the genomic region (e.g., as described in Stolc et al., 2005, Plant Mol. Biol. 59:137; Yuan et al., 2005, Science 309:626), Affymetrix Yeast Tiling Microarrays (YTM) provide complete and redundant coverage of the genome using a stepwise design in which probe sequences overlap such that each adjacent probe is shifted 5 bp relative to the reference sequence. The physical location of probes is randomized on the YTM thereby minimizing the confounding effect of any spatial bias in hybridization. Probe sequences cover 98.5% of the genome with no obvious bias in excluded genomic sequence (i.e., probes corresponding to repetitive regions such as telomeres and retrotransposons are represented). More than half (51.8%) of the genomic sequence is covered by 6 or more probes and only 11.9% is represented by 3 or fewer probes. For each 25-mer designed to perfectly match the reference sequence (i.e., perfectly-matched {PM} probes) there is a corresponding mismatched (MM) probe in which the 13th nucleotide has been replaced with an alternative base (i.e., A with T, G with C and vice versa). Thus, the microarray has approximately 2.6 million PM probes and approximately 2.6 million corresponding MM probes for a total of approximately 5.2 million probes on the array.

We modeled the decrease in probe intensity caused by a single SNP as a function of the SNP's position within the probe, the sequence surrounding it, the probe's G-C content and the hybridization intensity obtained using a non-polymorphic reference genome (i.e., that of strain FY3). The genomic sequences represented in the YTM tiled array represent a collection of sequences derived from strain S288C yeast cells and cells generated within a few generation from strain S288C. As demonstrated (e.g., using the methods described herein), at least several spontaneous mutations are likely to arise within the span of a few generations in a genome as large (i.e., ca. 12 Mb) as that of this yeast. For that reason, it is recognized that no yeast strain likely exists that has a genomic sequence 100% identical to that represented in the YTM array. Nonetheless, the genome of strain FY3 is believed to be as close to the array sequences that is practically feasible, likely differing by a relatively small-number (i.e., <a few dozen) of nucleotide residues.

To fit the model, we used a set of known SNPs that occur in strain RM11-1a (SNP sequences available at the World Wide Web site of the Broad Institute, for example), which were identified by direct comparison of its genomic sequence to that of the S288C strain reference genome. Sequences were aligned with the global alignment software MAVID and scanned for single nucleotide variants. That scanning process identified 86,443 potential SNPs. Of those, we eliminated from consideration any that were within 25 bp of another SNP, to ensure that we examined only probes that differed from the hybridized sample's DNA at a single base. To avoid potentially confounding effects of inaccurate or incomplete sequence, we further eliminated any sites where the RM11-1a sequence had a PHRED score less than 30, as well as any within 50 base pairs of an insertion or a deletion in the RM11-1a/S288C alignment. (PHRED refers to the well known software program used for base-calling in DNA sequence traces, and a score less than 30 indicates regions of significant sequence uncertainty.) In addition, we excluded probes that had more than 15 identical BLAST hits in the reference genome and those probes for which the difference in hybridization intensity of the PM−MM≦−0.05 (9,563 of 2,464,467 probes across the whole array) in the analysis of the strain FY3 reference genome sample.

This left a training set of 24,848 SNPs, which were overlapped by 123,016 probes on the tiling array.

We reserved a randomly-selected sample of 1000 of the identified SNPs for testing and fit the model to the remaining SNPs.

All intensity values were log₂ transformed and normalized for the purpose of training the model and performing SNP predictions. To normalize each hybridization we used a method similar to Li and Wong's set invariant normalization (Li et al., 2001, Proc. Natl. Acad. Sci. USA 98:31). We selected a subset of probes that appeared with high confidence to interrogate non-polymorphic regions of the sample. These were probes with intensity less than 1.5 standard deviations from their median value in five reference hybridizations. Use of this subset ensured that normalization eliminated only those intensity differences due to random experimental variation, and not true sequence differences between sample and reference. We then used a lowess data-smoothing procedure (the lowess function that is part of the R (lowess( )) statistical package, using a smoothing parameter of 0.05) to fit the difference between sample and reference measurements as a function of sample intensity. We used this function to normalize the intensities of all probes on the sample microarray.

Signal intensities from a single hybridization of RM11-1a genomic DNA were compared with the corresponding averages for five hybridizations performed using digested genomic DNA from strain FY3. We observed that a SNP's position within the probe is a strong predictor of its effect on signal intensity, with more central positions having a larger effect. This effect is visible in FIG. 1. The average intensity obtained for the reference DNA sample is a strong predictor of a probe's behavior in the presence of a SNP, mainly because the difference between the intensity resulting from binding with PM and MM probes is positively correlated with the signal decrease caused by a SNP, as shown in FIG. 2. The difference between PM and MM probe hybridization intensity can reflect multiple factors, each of which can affect hybridization efficiency. Such factors include cross-hybridization potential, the tendency to form secondary structures that impede hybridization, and the effect of a probe's specific sequence on the stability of duplex formation. It was also observed that the G-C content of a probe and the local sequence context of a potential mismatch, defined as the neighboring two nucleotides significant affected hybridization intensity. Taken together, these predictors explained 72% of the variance in signal change caused by the presence of a SNP. We used these data to fit a model that predicts the intensity of a probe in the presence of a specified SNP.

We modeled the difference between hybridization intensity for YTM hybridizations using strain RM11-1a DNA and strain FY3 reference DNA as:

Dᵢⱼₜ=αₜⱼ+βₜⱼ(GCᵢ)+γtj(PMᵢ−MMᵢ)+δₜⱼPMᵢ+Iᵢⱼₜ

where Dᵢⱼₜ is the signal difference for perfect match probe i overlapping a SNP at position j within the probe, with nucleotide triplet t including and flanking j. GCᵢ is the G-C content of probe i, and PMᵢ and MMᵢ are the intensities of perfect match and mismatch probes for the reference sequence. Iᵢⱼₜ represents the two- and three-way interaction terms among the three covariates, omitted here for clarity (these terms are clear to a skilled artisan in this field and do not need to be explicitly listed here). α, β, γ, And δ are constants for which values were estimated by fitting experimental data from the reference (FY3) genome fragments to this model.

We fit the model and estimated parameters using the statistical analysis package R (available, for example, at the World Wide Web site of the R Foundation for Statistical Computing). To check against over-fitting attributable to the model's large size, we estimated its 4608 parameters using-only half of the probes in the training set and then used these values to predict signal changes for the other half. Observed and predicted values for the training set were correlated with an R² value of 0.73, while those for the test set were correlated with an R² of 0.68, demonstrating that the model predicted novel data nearly as well as it did the data used for fitting.

We used the model to determine, for each base in the genome, the expected signal intensities of all overlapping probes under two distinct hypotheses: i) the base in the hybridized sequence is identical to the reference sequence or ii) different from it: In the non-polymorphic case, the expected intensity μₙᵢ of probe i was estimated as the observed mean intensity of i over the five FY3 hybridizations. In the polymorphic case, the expected intensity μₚᵢ was estimated as μₙᵢ−Dᵢⱼₜ. For both cases, the intensity variance σᵢ² was estimated based on the sample variance over the FY3 hybridizations. We assigned each probe the average variance across a set of 501 probes with a similar minimum intensity (Comander et al., 2004, BMC Genomics 5:17). Assuming a normal distribution in either case, these values can then be used to generate a log likelihood ratio that the observed intensities xᵢ for the corresponding location k in a given sample indicates a polymorphic site or a non-polymorphic site:

$${L}_{k} = ( {log}_{10} e ) {\sum }_{i} \frac{{( {x}_{i} - {μ}_{ni} )}^{2} - {( {x}_{i} - {μ}_{pi} )}^{2}}{2 {σ}_{i}^{2}}$$

In this equation, we have taken the log of the likelihood ratio, summed over i probes overlapping the site, and converted to log₁₀. Positive values of Lₖ (hereafter referred to as the prediction signal) indicate a greater likelihood that site k is polymorphic than non-polymorphic, with higher values corresponding to increased confidence in this conclusion. This score is highly sensitive to underestimates of σᵢ², if the observed intensity of a probe is far from either μₙᵢ or μₚᵢ. To avoid misleading extremes introduced by this sensitivity, the lowest variance among the probes overlapping a site was increased to the value of the second lowest variance.

Probes were eliminated from the score calculation if they made a negative contribution to the prediction signal despite having intensities more than two standard deviations below μₙᵢ. We implemented this algorithm in a program called SNPscanner written in the C++ computer programming language. The algorithm is referred to herein as the SNPscanner algorithm.

Once L was calculated for every site in the genome, potential polymorphic sites were identified as those nucleotides having a prediction signal greater than a threshold value of 0. The presence of a SNP results in positive prediction signal for adjacent non-polymorphic bases, yielding a characteristic region of elevated prediction signal delimited by at least 5 consecutive nucleotides with a prediction signal below zero. A region corresponding to a predicted SNP included drops below threshold, as long as these extended for no more than four bases. The predicted position of the SNP was defined as the base in the region with the maximum score L.

Although this approach yielded a high true positive rate, we sought to minimize the number of false positives by imposing additional criteria for filtering SNP predictions on a genome-wide scale. We considered any SNPs covered by a single probe on the array to be unreliably measured and discarded any predictions that were not based on data from at least two probes. In addition, we applied a voting scheme requiring that more than one probe must individually predict the presence of a SNP at position k with a prediction signal >5 and the number of probes that individually predict the presence of a SNP at position k must be greater than the number of probes that predict position k is non-polymorphic with a prediction signal <−5. In this voting scheme, prediction signals between −5 and 5 are given a weight of 0. Finally, predictions were removed if the region above the signal threshold associated with a peak did not extend for at least 6 nucleotides. This set of heuristic filters is not the only set that can be used. A skilled artisan in this field understands that a variety of filters can be used including, for example, the number of probes covering the nucleotide residue being predicted, the fraction of probes that cover a nucleotide residue that predict occurrence of a polymorphism at the residue, and the length of the region of positive prediction.

We wished to extend our approach to allow the prediction of SNPs that differ between two related non-reference strains, A and A′, using data from a single hybridization experiment of each DNA. To do this we first predicted polymorphisms using the SNPscanner algorithm and filtered the resulting predictions for each sample using the criteria described above. We compared predictions for each sample and identified unique regions of positive prediction signal in A′ that were absent in the corresponding region of A. In order to call a SNP present in A′ and absent in A, we required that the prediction signal remain ≦0 for the entire corresponding region of A for which the prediction signal in A′≧0 and reached a maximum of at least 5.

We observed that, in addition to being sensitive to predicting the presence of SNPs, the SNPscanner algorithm accurately detected the presence of large deletions, which are characterized by large continuous regions in which the prediction signal remains greater than zero. Generally, the width of a region with prediction signal greater than 0 in the presence of a SNP was 18-22 nucleotides. Although it remains possible that a larger continuous region with prediction values greater than zero is indicative of a large number of localized polymorphisms, we considered regions in which the prediction signal remained above zero for >50 nucleotides as likely evidence of the presence of a deletion.

Model Validation

We tested the performance of our algorithm using a set of 1,000 high quality SNPs from RM11-1a that had been excluded when training the model. We confirmed that 981 SNPs were represented by at least one probe on the YTM and excluded the other 19 SNPs from further analysis. At a prediction signal >0 we detected 944 of the 981 (96.2%) SNPs. We compared our false negative rate to our false discovery rate (FDR) by using the SNPscanner algorithm to predict SNPs on hybridization data from FY3 DNA not included in the training data. At a prediction signal >0 we called 23,026 putative SNPs in FY3 (FIG. 3). By increasing our threshold to a prediction signal >5 we were able to reduce the number of false positives across the genome to 7 (approximately 1 false positive per 2 million bp) while retaining predictions for 801 (81.7%) SNPs in RM-11-1a genomic DNA. By filtering these raw SNPscanner data using the empirically-derived criteria described above, we were able to reduce the number of false positives to 0 whilst retaining 77.5% (760 of 981) of real SNPs.

We investigated the level of precision with which we were able to call polymorphic sites. Of the 760 SNPs predicted at a prediction signal of 5, 347 (45.6%) were predicted at the precise polymorphic base pair. An additional 316 (41.5%) SNPs were called 1 or 2 bases away from the exact location; i.e., our algorithm correctly predicts the location of a SNP within two base pairs 87.1% of the time.

Genome-Wide Mutation Prediction Applied to Positional Cloning

Yeast strain CP1AB is a homozygous diploid strain that is derived from an S288C background. Despite its presumed isogenicity with the S288C-derived strain, FY3, CP1AB has a growth deficiency on acetate, and genetic crosses to FY3 indicated mendelian segregation of the phenotype. This growth defect has been mapped to a 50-kilobase (kb) centromeric region of chromosome XVI, but could not be further localized due to the low recombination rate in this region (Brauer et al., 2006, Genetics 173:1813).

CP1AB has undergone experimental evolution for >200 generations under glucose limitation, yielding a number of independently evolved strains (Ferea et al., 1999, Proc. Natl. Acad. Sci. USA 96:9721). One of these, E2, is once again able to grow on acetate and, when sporulated, shows 2:2 segregation of the acetate growth defect, indicating an acquired dominant mutation (Dunham et al., 2002, Proc. Natl. Acad. Sci. USA 99:16144). Crosses with FY3 of an E2 spore able to grow on acetate resulted in approximately 100% of segregants being able to grow on acetate. These results generated two expectations: a sequence difference between CP1AB and FY3 corresponding to the cause of the acetate phenotype in the former, and a second difference, either a true reversion or a closely linked sequence change, that causes reversal of the phenotype in E2.

When we analyzed genomic DNA from these strains using YTMs and the SNPscanner algorithm, those expectations were unambiguously met. Both CP1AB and the E2 spore differed from FY3 at nucleotide position chrXVI:549440. This site falls within the AEP3 gene, which encodes a mitochondrial protein that stabilizes the mRNA encoding subunits 6 and 8 of the ATP synthase complex. Disruption of AEP3 has been reported to result in a growth defect on non-fermentable carbon sources (Ellis et al., 2004, J. Biol. Chem. 279:15728). We sequenced AEP3 in CP1AB and the E2 spore and identified a mutation, chrXVI:549441A→C in CP1AB (Table 3) that results in a Q320P replacement at an amino acid residue conserved in all Saccharomyces sensu stricto yeast species. Sequence analysis of the E2 spore identified both this mutation in addition to an adjacent mutation, chrXVI:549440C→A that results in a P320T replacement and a second mutation chrXVI:549450A→G resulting in a E323V replacement three codons downstream. One or both of these mutations must be an intragenic suppressor of the mutation that originated on the CP1AB allele. Subsequent sequence analysis of an additional strain evolved from CP1AB, strain E3, that also segregated an acetate growth phenotype as a mendelian trait that was mapped to the same locus (Dunham et al., 2002, Proc. Natl. Acad. Sci. USA 99:16144), identified a mutation that was a true reversion to the original S288C sequence (Table 3).

The disclosure of every patent, patent application, and publication cited herein is hereby incorporated herein by reference in its entirety.

While this subject matter has been disclosed with reference to specific embodiments, it is apparent that other embodiments and variations can be devised by others skilled in the art without departing from the true spirit and scope of the subject matter described herein. The appended claims include all such embodiments and equivalent variations.

1. A method of assessing the likelihood that a polymorphism occurs at a nucleotide position in a first genome, relative to the same nucleotide position in a corresponding reference genome, the method comprising
contacting, under hybridizing conditions, fragments of the first genome with a plurality of polynucleotide probes, wherein the probes are completely complementary to different, but overlapping, portions of the reference genome that include the nucleotide position;
assessing the hybridization intensity for each of the probes with the fragments;
for each probe, comparing the assessed hybridization intensity with the predicted hybridization intensity, wherein the predicted hybridization intensity for each probe is calculated based on hybridization intensity of fragments of the reference genome with the probes; and
combining the comparisons for each probe to yield an estimate the polymorphism occurs at the nucleotide position in the first genome.

2. The method of claim 1, wherein the predicted hybridization intensity for each probe is further calculated based on assumed occurrence of a non-complementary nucleotide residue at the residue corresponding to the nucleotide position within the probe.

3. The method of claim 1, wherein the likelihood that a polymorphism occurs is assessed at each of a plurality of closely-spaced nucleotide positions in the first genome and wherein a polymorphism is predicted to occur at the position having the highest estimate that the polymorphism occurs at or near that position.

4. The method of claim 1, wherein the likelihood that a polymorphism occurs is assessed at each of a plurality of consecutive nucleotide positions in the first genome and wherein a polymorphism is predicted to occur at the position having the highest estimate that the polymorphism occurs at that position.

5. The method of claim 4, wherein the likelihood that a polymorphism occurs is assessed at each of at least 10 consecutive nucleotide positions in the first genome.

6-7. (canceled)

8. The method of claim 1, wherein the fragments of the first genome are prepared by enzymatic digestion.

9. (canceled)

10. The method of claim 1, wherein the fragments are prepared by amplification of portions of the first genome.

11. The method of claim 1, wherein substantially all of the fragments of the first genome have a length in the range from 10 to 60 nucleotide residues.

12. The method of claim 1, wherein hybridization intensity is assessed by assessing fluorescence of a dye associated with the probes.

13. (canceled)

14. The method of claim 1, wherein each of the probes is bound to a support at a discrete location.

15. The method of claim 1, wherein each of the probes is bound to a discrete support.

16. The method of claim 1, wherein the fragments are bound to a support at discrete locations.

17. The method of claim 1, wherein the predicted hybridization intensity for each probe is calculated based both on the occurrence of a non-complementary nucleotide residue at the residue corresponding to the nucleotide position within the probe and on the occurrence of a complementary nucleotide residue at the residue corresponding to the nucleotide position within the probe.

18. The method of claim 1, wherein the predicted hybridization intensity for each probe is corrected for the hybridization intensity variance observed for a plurality of hybridizations performed using fragments of the reference genome.

19. (canceled)

20. The method of claim 1, wherein the comparisons of assessed and predicted hybridization intensities are made by calculating a prediction signal (Lₖ) from the values obtained for each of i probes, wherein
$${L}_{k} = ( {log}_{10} e ) {\sum }_{i} \frac{{( {x}_{i} - {μ}_{ni} )}^{2} - {( {x}_{i} - {μ}_{pi} )}^{2}}{2 {σ}_{i}^{2}}$$
wherein xᵢ is the assessed hybridization intensity for an individual probe, μₙᵢ is the predicted hybridization intensity for the individual probe when no polymorphism occurs at the nucleotide position, μₚᵢ is the predicted hybridization intensity for the individual probe when a polymorphism occurs at the nucleotide position, and σᵢ is the intensity variance for the probe, whereby a positive value of Lₖ is indicative that a polymorphism occurs at the nucleotide position and a negative value of Lₖ is indicative that a polymorphism does not occur at the nucleotide position.

21. The method of claim 20, wherein μₙᵢ is assessed based on hybridization intensity values observed when fragments of the reference genome are hybridized with the probe.

22. The method of claim 21, wherein σᵢ is assessed based on the variance in hybridization intensity values observed when fragments of the reference genome are hybridized with the probe.

23. The method of claim 22, wherein μₚᵢ is calculated for each of the i probes using the formula μₚᵢ=μₙᵢ−Dᵢ, wherein
Dᵢ=α+β(GCᵢ)+γ(PMᵢ−MMᵢ)+δPMᵢ
wherein Dᵢ is the signal difference for a completely complementary probe overlapping a polymorphism, GCᵢ is the G-C content of the probe, PMᵢ is the hybridization intensity of the probe when hybridized with a completely complementary polynucleotide, MMᵢ is the hybridization intensity of the probe when hybridized with a complementary polynucleotide having a single mismatched base at its center, and each of α, β, γ, and δ is a constant derived from statistical fitting of data obtained from hybridization of the probe with fragments of the reference genome.

24. The method of claim 1, wherein the sequence of the reference genome is substantially known.

25-34. (canceled)

35. The method of claim 1, wherein the probes have lengths in the range from 15 to nucleotide residues.

36. The method of claim 1, wherein the assessed and predicted hybridization intensities are not compared for probes for which the probe residue corresponding to the nucleotide residue occurs within 5 residues from an end of the probe.

37. A method of assessing the location of one or more polymorphisms in a first genome, relative to a corresponding reference genome, the method comprising:
i) contacting, under hybridizing conditions, fragments of the first genome with a plurality of polynucleotide probes, wherein the probes are completely complementary to different, but overlapping, portions of the reference genome;
ii) assessing the hybridization intensity for each of the probes with the fragments;
iii) for each of a plurality of genomic locations which are overlapped by multiple probes:
a) for each probe that overlaps the location, comparing the assessed hybridization intensity with the predicted hybridization intensity, wherein the predicted hybridization intensity for each probe is calculated based on hybridization intensity of fragments of the reference genome with the probes; and
b) combining the comparisons for each probe to yield an estimate that the polymorphism occurs at the location; and
iv) comparing the estimates to identify where, within the overlapping portions, a polymorphism occurs, whereby the polymorphism is predicted to occur at the nucleotide residue for which the estimate is highest.

38. The method of claim 37, wherein the predicted hybridization intensity for each probe is further calculated based on assumed occurrence of a non-complementary nucleotide residue at the residue corresponding to the nucleotide position within the probe.

39. The method of claim 37, wherein the overlapping portions are consecutive and encompass at least 100 base pairs.

40. The method of claim 37, wherein the overlapping portions are consecutive and encompass at least several hundred base pairs.

41. The method of claim 37, wherein the overlapping portions encompass substantially all of the reference genome.

42. The method of claim 37, wherein the genomic locations include at least 25 consecutive nucleotide residues.

43-47. (canceled)

48. The method of claim 37, wherein substantially all of the fragments of the first genome have a length in the range from 10 to 60 nucleotide residues.

49-51. (canceled)

52. The method of claim 37, wherein the predicted hybridization intensity for each probe is calculated based both on the occurrence of a non-complementary nucleotide residue at the residue corresponding to the nucleotide position within the probe and on the occurrence of a complementary nucleotide residue at the residue corresponding to the nucleotide position within the probe.

53. The method of claim 37, wherein the predicted hybridization intensity for each probe is corrected for the hybridization intensity variance observed for a plurality of hybridizations performed using fragments of the reference genome.

54. (canceled)

55. The method of claim 37, wherein the comparisons of assessed and predicted hybridization intensities are made by calculating a prediction signal (Lₖ) from the values obtained for each of i probes, wherein
$${L}_{k} = ( {log}_{10} e ) {\sum }_{i} \frac{{( {x}_{i} - {μ}_{ni} )}^{2} - {( {x}_{i} - {μ}_{pi} )}^{2}}{2 {σ}_{i}^{2}}$$
wherein xᵢ is the assessed hybridization intensity for an individual probe, μₙᵢ is the predicted hybridization intensity for the individual probe when no polymorphism occurs at the nucleotide position, μₚᵢ is the predicted hybridization intensity for the individual probe when a polymorphism occurs at the nucleotide position, and σᵢ is the intensity variance for the probe, whereby a positive value of Lₖ is indicative that a polymorphism occurs at the nucleotide position and a negative value of Lₖ is indicative that a polymorphism does not occur at the nucleotide position.

56. The method of claim 55, wherein μₙᵢ is assessed based on hybridization intensity values observed when fragments of the reference genome are hybridized with the probe.

57. The method of claim 56, wherein σᵢ is assessed based on the variance in hybridization intensity values observed when fragments of the reference genome are hybridized with the probe.

58. The method of claim 57, wherein μₚᵢ is calculated for each of the i probes using the formula μₚᵢ=μₙᵢ−Dᵢ, wherein
Dᵢ=α+β(GCᵢ)+γ(PMᵢ−MMᵢ)+δPMᵢ
wherein Dᵢ is the signal difference for a completely complementary probe overlapping a polymorphism, GCᵢ is the G-C content of the probe, PMᵢ is the hybridization intensity of the probe when hybridized with a completely complementary polynucleotide, MMᵢ is the hybridization intensity of the probe when hybridized with a complementary polynucleotide having a single mismatched base at its center, and each of α, β, γ, and δ is a constant derived from statistical fitting of data obtained from hybridization of the probe with fragments of the reference genome.

59-69. (canceled)

70. The method of claim 37, wherein the probes have lengths in the range from 10 to 60 nucleotide residues.

71. The method of claim 37, wherein the assessed and predicted hybridization intensities are not compared for probes for which the probe residue corresponding to the nucleotide residue occurs within 5 residues from an end of the probe.

72. A kit for assessing occurrence of one or more polymorphisms in a first genome, relative to a corresponding reference genome, the kit comprising:
i) an array comprising a plurality of polynucleotide probes attached at discrete known locations on a support, wherein the probes are completely complementary to different, but overlapping, portions of the reference genome;
ii) a digital storage medium including an algorithm for comparing assessed hybridization intensity values for the probes with predicted hybridization intensity values, whereby occurrence of one or more polymorphisms in the first genome can be assessed.

73-76. (canceled)
